(19) United States
(12) Patent Application Publication
(10) Pub. No.: US 2019/0279751 A1
(43) Pub. Date: Sep. 12, 2019

(54) MEDICAL DOCUMENT CREATION SUPPORT APPARATUS, METHOD, AND PROGRAM

(71) Applicant: FUJIFILM Corporation, Tokyo (JP)

(72) Inventors: Keigo NAKAMURA, Tokyo (JP); Yohei MOMOKI, Kanagawa (JP)

(73) Assignee: FUJIFILM Corporation, Tokyo (JP)

(21) Appl. No.: 16/277,330

(22) Filed: Feb. 15, 2019

(30) Foreign Application Priority Data
Mar. 6, 2018 (JP) 2018-040203

(51) Int. Cl. G16H 15/00; G16H 30/20; G06F 17/27; G10L 15/26

(52) U.S. Cl. CPC G16H 15/00; G10L 15/26; G06F 17/2775; G16H 30/20

(57) ABSTRACT

An input receiving unit receives an input of a keyword indicating the findings based on a displayed medical image. An analysis result acquisition unit acquires an analysis result of the medical image. A document creation unit creates a medical document relevant to the medical image based on the keyword and the analysis result.

### CROSS REFERENCE TO RELATED APPLICATIONS

The present application claims priority under 35 U.S.C. § 119 to Japanese Patent Application No. 2018-040203 filed on Mar. 6, 2018. The above application is hereby expressly incorporated by reference, in its entirety, into the present application.

### BACKGROUND

### Field of the Invention

The present invention relates to a medical document creation support apparatus, method, and program for supporting the creation of a medical document, such as an interpretation report.

### Related Art

In recent years, advances in medical apparatuses, such as computed tomography (CT) apparatuses and magnetic resonance imaging (MRI) apparatuses, have enabled image diagnosis using high-resolution medical images with higher quality. In particular, since the region of a lesion can be accurately specified by image diagnosis using CT images, MRI images, and the like, appropriate treatment can be performed based on the specified result.

A medical image is analyzed by computer-aided diagnosis (CAD) using a discriminator learned by deep learning or the like, regions, positions, volumes, and the like of lesions included in the medical image are extracted, and these are acquired as the analysis result. The analysis result generated by analysis processing in this manner is stored in a database so as to be associated with examination information, such as a patient name, gender, age, and a modality that has acquired the medical image, and provided for diagnosis. At this time, a technician in a radiology department or the like, who has acquired the medical image, determines a radiologist according to the medical image, and notifies the radiologist that the medical image and the result of analysis by the CAD are present. The radiologist interprets the medical image with reference to the transmitted medical image and analysis result and creates an interpretation report at his or her own interpretation terminal.

Various methods have been proposed to support the creation of a medical document in a case where a medical document, such as an interpretation report, is created. For example, a method has been proposed in which a finding database search using a certain input keyword is performed to extract findings in which the keyword is used, the extracted findings are displayed as candidate findings on the display screen in the order based on the matching rate of the keyword, and a finding sentence arbitrarily designated in the displayed candidate findings is posted to findings under creation (refer to JP2003-122848A). In addition, a method has been proposed in which templates of sentences to be used in an interpretation report is stored on the interpretation report creation screen, keywords searched for based on the contents input by voice are displayed together with images, and processing for embedding the keywords in the template that is a model of sentences is performed to complete the sentences of the interpretation report (refer to JP2005-027978A). In addition, a method has been proposed in which, in a case where contents to be described in a diagnosis report are input by voice, the voice is converted into a character string and the character string data is analyzed, and the diagnosis report is created based on the contents of words, sentences, and the like stored in a report information storage unit (refer to JP2004-230001A).

However, in the case of creating an interpretation report using the methods described in JP2003-122848A, JP2005-027978A, and JP2004-230001A, the radiologist needs to carefully interpret a medical image and input all the findings obtained from the medical image using a keyword or the like. For this reason, the burden on the radiologist who creates an interpretation report is large.

### SUMMARY

The invention has been made in view of the above circumstances, and it is an object of the invention to reduce the burden on the operator, such as a radiologist, in a case where a medical document, such as an interpretation report on a medical image, is created.

A medical document creation support apparatus according to the invention comprises: an input receiving unit that receives an input of a keyword indicating findings based on a displayed medical image; an analysis result acquisition unit that acquires an analysis result of the medical image; and a document creation unit that creates a medical document relevant to the medical image based on the keyword and the analysis result.

In the medical document creation support apparatus according to the invention, the analysis result acquisition unit may acquire the analysis result by analyzing the medical image.

In the medical document creation support apparatus according to the invention, the analysis result may include contents indicating findings based on the medical image, and the document creation unit may create the medical document based on the keyword and the contents.

The medical document creation support apparatus according to the invention may further comprise a display control unit that displays the medical image and the medical document on a display unit.

In the medical document creation support apparatus according to the invention, the input receiving unit may receive an input of an additional keyword for the displayed medical document, and the document creation unit may create the medical document again based on the additional keyword in addition to the keyword and the analysis result.

In the medical document creation support apparatus according to the invention, the document creation unit may create a plurality of medical document candidates for one medical image, the display control unit may display the plurality of medical document candidates on the display unit, and the document creation unit may further create the medical document based on a medical document candidate selected from the plurality of medical document candidates.

In the medical document creation support apparatus according to the invention, the medical document may be an interpretation report on the medical image.

In the medical document creation support apparatus according to the invention, the medical document may include a link to the medical image.

In the medical document creation support apparatus according to the invention, the input receiving unit may receive an input of a keyword of voice indicating the findings and convert the keyword of the voice into text.

A medical document creation support method according to the invention comprises: receiving an input of a keyword indicating findings based on a displayed medical image; acquiring an analysis result of the medical image; and creating a medical document relevant to the medical image based on the keyword and the analysis result.

In addition, a program causing a computer to execute the medical document creation support method according to the invention may be provided.

Another medical document creation support apparatus according to the invention comprises: a memory that stores commands to be executed by a computer; and a processor configured to execute the stored commands. The processor executes: a process of receiving an input of a keyword indicating findings based on a displayed medical image; a process of acquiring an analysis result of the medical image; and a process of creating a medical document relevant to the medical image based on the keyword and the analysis result.

According to the invention, an input of a keyword indicating the findings based on the displayed medical image is received, the analysis result of the medical image is acquired, and a medical document relevant to the medical image is created based on the keyword and the analysis result. In this manner, in a case where the operator inputs only a keyword, a medical document including both the analysis result and the operator's recognition is created. Therefore, according to the invention, it is possible to reduce the burden on the operator in a case where a medical document is created. As a result, the operator can efficiently create a medical document. Since the manner of expressing a medical document differs depending on the operator who creates the medical document, there is a variation in a document for each operator. In this manner, in a case where there is a variation in a document, it may be difficult to read a document or it may be difficult to reuse a document. According to the invention, since it is possible to suppress a variation in a created medical document, it is possible to create a medical document that is easy to read and easy to reuse.

### BRIEF DESCRIPTION OF THE DRAWINGS

FIG. 1 is a diagram showing the schematic configuration of a medical information system to which a medical document creation support apparatus according to an embodiment of the invention is applied.

FIG. 2 is a diagram showing the schematic configuration of the medical document creation support apparatus according to the present embodiment.

FIG. 3 is a diagram showing the schematic configuration of a recurrent neural network.

FIG. 4 is a diagram showing an interpretation report creation screen.

FIG. 5 is a diagram showing an interpretation report creation screen.

FIG. 6 is a flowchart showing a process performed in the present embodiment.

FIG. 7 is a diagram showing an interpretation report creation screen.

### DETAILED DESCRIPTION

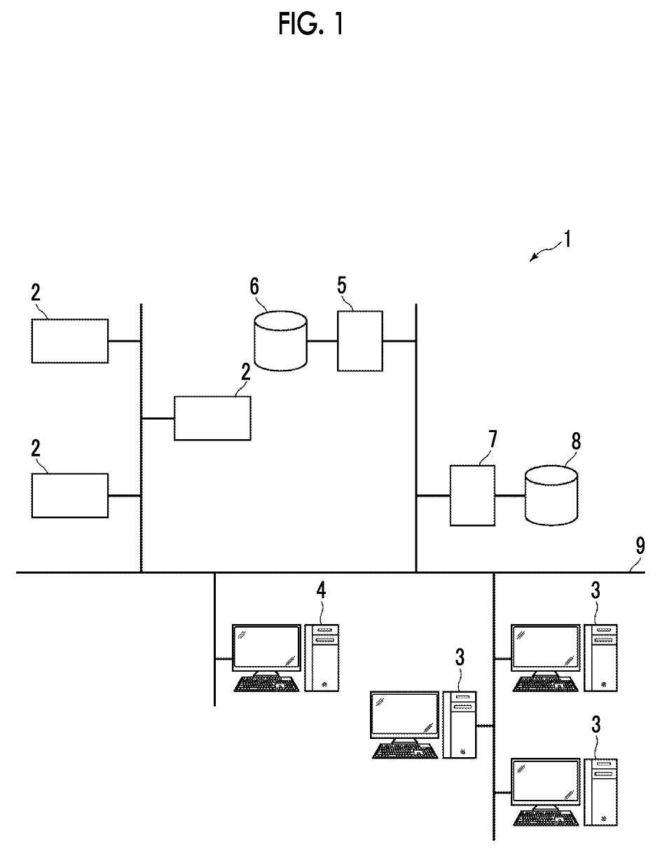

Hereinafter, an embodiment of the invention will be described with reference to the accompanying diagrams. FIG. 1 is a diagram showing the schematic configuration of a medical information system to which a medical document creation support apparatus according to an embodiment of the invention is applied. A medical information system 1 shown in FIG. 1 is a system for performing imaging of an examination target part of a subject, storage of a medical image acquired by imaging, interpretation of a medical image by a radiologist and creation of an interpretation report, and viewing of an interpretation report by a doctor in a medical department of a request source and detailed observation of a medical image to be interpreted, based on an examination order from a doctor in a medical department using a known ordering system. As shown in FIG. 1, the medical information system 1 is configured to include a plurality of modalities (imaging apparatuses) 2, a plurality of interpretation workstations (WS) 3 that are interpretation terminals, a medical department workstation (WS) 4, an image server 5, an image database 6, an interpretation report server 7, and an interpretation report database 8 that are communicably connected to each other through a wired or wireless network 9.

Each apparatus is a computer on which an application program for causing each apparatus to function as a component of the medical information system 1 is installed. The application program is recorded on a recording medium, such as a digital versatile disc (DVD) or a compact disc read only memory (CD-ROM), and distributed, and is installed onto the computer from the recording medium. Alternatively, the application program is stored in a storage device of a server computer connected to the network 9 or in a network storage so as to be accessible from the outside, and is downloaded and installed onto the computer as necessary.

The modality 2 is an apparatus that generates a medical image showing a diagnosis target part by imaging the diagnosis target part of the subject. Specifically, the modality 2 is a simple X-rays imaging apparatus, a CT apparatus, an MRI apparatus, a positron emission tomography (PET) apparatus, and the like. A medical image generated by the modality 2 is transmitted to the image server 5 and stored therein.

The interpretation WS 3 includes the medical document creation support apparatus according to the present embodiment. The configuration of the interpretation WS 3 will be described later.

The medical department WS 4 is a computer used by a doctor in a medical department to observe the details of an image, view an interpretation report, create an electronic medical record, and the like, and is configured to include a processing device, a display device such as a display, and an input device such as a keyboard and a mouse. In the medical department WS 4, each process, such as creation of a patient's medical record (electronic medical record), sending a request to view an image to the image server 5, display of an image received from the image server 5, automatic detection or highlighting of a lesion-like portion in an image, sending a request to view an interpretation report to the interpretation report server 7, and display of an interpretation report received from the interpretation report server 7, is performed by executing a software program for each process.

The image server 5 is obtained by installing a software program for providing a function of a database management system (DBMS) on a general-purpose computer. The image server 5 comprises a storage for an image database 6. This storage may be a hard disk device connected to the image server 5 by a data bus, or may be a disk device connected to a storage area network (SAN) or a network attached storage (NAS) connected to the network 9. In a case where the image server 5 receives a request to register a medical image from the modality 2, the image server 5 registers the medical image in the image database 6 in a format for a database.

Image data and accessory information of medical images acquired by the modality 2 are registered in the image database 6. The accessory information includes, for example, an image ID for identifying each medical image, a patient identification (ID) for identifying a subject, an examination ID for identifying an examination, a unique ID (UID: unique identification) allocated for each medical image, examination date and examination time at that the medical image is generated, the type of a modality used in an examination for acquiring a medical image, patient information such as patient's name, age, and gender, an examination part (imaging part), imaging information (an imaging protocol, an imaging sequence, an imaging method, imaging conditions, the use of a contrast medium, and the like), and information such as a series number or a collection number in a case where a plurality of medical images are acquired in one examination.

In a case where a viewing request from the interpretation WS 3 is received through the network 9, the image server 5 searches for a medical image registered in the image database 6 and transmits the searched medical image to the interpretation WS 3 that is a request source.

The interpretation report server 7 has a software program for providing a function of a database management system to a general-purpose computer. In a case where the interpretation report server 7 receives a request to register an interpretation report from the interpretation WS 3, the interpretation report server 7 registers the interpretation report in the interpretation report database 8 in a format for a database. In a case where a request to search for an interpretation report is received, the interpretation report is searched for from the interpretation report database 8.

In the interpretation report database 8, for example, an interpretation report is registered in which information, such as an image ID for identifying a medical image to be interpreted, a radiologist ID for identifying an image diagnostician who performed the interpretation, a lesion name, position information of a lesion, findings, and the certainty of findings, is recorded.

The network 9 is a wired or wireless local area network that connects various apparatuses in a hospital to each other. In a case where the interpretation WS 3 is installed in another hospital or clinic, the network 9 may be configured to connect local area networks of respective hospitals through the Internet or a dedicated circuit. In any case, it is preferable that the network 9 is configured to be able to realize high-speed transmission of medical images, such as an optical network.

Hereinafter, the interpretation WS 3 according to the present embodiment will be described in detail. The interpretation WS 3 is a computer used by a radiologist of a medical image to interpret the medical image and create the interpretation report, and is configured to include a processing device, a display device such as a display, and an input device such as a keyboard and a mouse. In the interpretation WS 3, each process, such as making a request to view a medical image to the image server 5, various kinds of image processing on a medical image received from the image server 5, display of a medical image, analysis processing on a medical image, highlighting of a medical image based on the analysis result, creation of an interpretation report based on the analysis result, support for the creation of an interpretation report, making a request to register an interpretation report and a request to view an interpretation report to the interpretation report server 7, and display of an interpretation report received from the interpretation report server 7, is performed by executing a software program for each process. Since processes other than the process performed by the medical document creation support apparatus of the present embodiment, among these processes, are performed by a known software program, the detailed description thereof will be omitted herein. The processes other than the process performed by the medical document creation support apparatus of the present embodiment may not be performed in the interpretation WS 3, and a computer that performs the processes may be separately connected to the network 9, and requested processing on the computer may be performed according to a processing request from the interpretation WS 3.

The interpretation WS 3 includes the medical document creation support apparatus according to the present embodiment. Therefore, a medical document creation support program according to the present embodiment is installed on the interpretation WS 3. The medical document creation support program is recorded on a recording medium, such as a DVD or a CD-ROM, and distributed, and is installed onto the interpretation WS 3 from the recording medium. Alternatively, the medical document creation support program is stored in a storage device of a server computer connected to the network or in a network storage so as to be accessible from the outside, and is downloaded and installed onto the interpretation WS 3 as necessary.

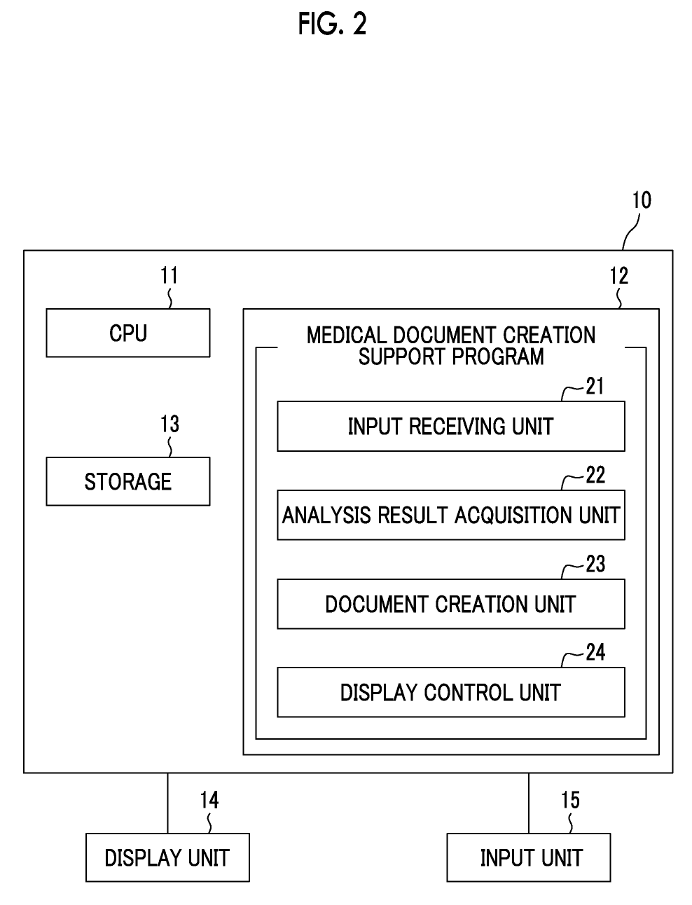

FIG. 2 is a diagram showing the schematic configuration of the medical document creation support apparatus according to the embodiment of the invention that is realized by installing the medical document creation support program. As shown in FIG. 2, a medical document creation support apparatus 10 comprises a central processing unit (CPU) 11, a memory 12, and a storage 13 as the configuration of a standard computer. A display device (hereinafter, referred to as a display unit) 14, such as a liquid crystal display, and an input device (hereinafter, referred to as an input unit) 15, such as a keyboard and a mouse, are connected to the medical document creation support apparatus 10. The input unit 15 may comprise a microphone to receive a voice input. In the present embodiment, it is assumed that the operator's voice input is received.

The storage 13 is a storage device, such as a hard disk or a solid state drive (SSD). Medical images and various kinds of information including information necessary for processing of the medical document creation support apparatus 10, which are acquired from the image server 5 through the network 9, are stored in the storage 13.

A medical document creation support program is stored in the memory 12. As processing to be executed by the CPU 11, the medical document creation support program defines: input reception processing for receiving an input of a keyword indicating findings based on a displayed medical image; analysis result acquisition processing for acquiring an analysis result of the medical image; document creation processing for creating a medical document relevant to the medical image based on the keyword and the analysis results; and display control processing for displaying the medical image and the medical document on a display unit 14.

The CPU 11 executes these processes according to the medical document creation support program, so that the computer functions as an input receiving unit 21, an analysis result acquisition unit 22, a document creation unit 23, and a display control unit 24. In the present embodiment, the CPU 11 executes the function of each unit according to the medical document creation support program. However, as a general-purpose processor that executes software to function as various processing units, a programmable logic device (PLD) that is a processor whose circuit configuration can be changed after manufacturing, such as a field programmable gate array (FPGA), can be used in addition to the CPU 11. Alternatively, the processing of each unit may also be executed by a dedicated electrical circuit that is a processor having a circuit configuration designed exclusively to execute specific processing, such as an application specific integrated circuit (ASIC).

One processing unit may be configured by one of various processors, or may be a combination of two or more processors of the same type or different types (for example, a combination of a plurality of FPGAs or a combination of a CPU and an FPGA). Alternatively, a plurality of processing units may be configured by one processor. As an example of configuring a plurality of processing units using one processor, first, as represented by a computer, such as a client and a server, there is a form in that one processor is configured by a combination of one or more CPUs and software and this processor functions as a plurality of processing units. Second, as represented by a system on chip (SoC) or the like, there is a form of using a processor that realizes the function of the entire system including a plurality of processing units with one integrated circuit (IC) chip. Thus, various processing units are configured by using one or more of the above-described various processors as a hardware structure.

More specifically, the hardware structure of these various processors is an electrical circuit (circuitry) in the form of a combination of circuit elements, such as semiconductor elements.

The input receiving unit 21 is an interface for receiving an input of a keyword input by the operator from the input unit 15. In the present embodiment, the operator performs voice input. Therefore, the input receiving unit 21 according to the present embodiment also has a function of converting the input voice into text.

The analysis result acquisition unit 22 acquires an analysis result on a disease or the like included in a medical image by analyzing the medical image. For this purpose, the analysis result acquisition unit 22 comprises a discriminator that is machine-learned to determine whether or not each pixel (voxel) in a medical image indicates a lesion and to determine the type of a lesion. In the present embodiment, the discriminator is a neural network deep-learned so as to be able to classify a plurality of types of lesions included in a medical image. The discriminator in the analysis result acquisition unit 22 is learned so as to output a probability that each pixel (voxel) in a medical image is each of a plurality of lesions in a case where the medical image is input. The discriminator determines a lesion, which exceeds a predetermined threshold value and has a maximum probability, for a certain pixel, and determines that the pixel is a pixel of a lesion for which the pixel has been determined.

The discriminator may be configured to include, for example, a support vector machine (SVM), a convolutional neural network (CNN), and a recurrent neural network (RNN), in addition to the deep-learned neural network.

The analysis result acquisition unit 22 generates an analysis result including the type of the lesion, the position of the lesion, and the size of the lesion, as contents indicating the findings of the medical image using the determination result of the discriminator described above. For example, in a case where a medical image includes a lung field, in a case where a lesion is found in the lung field, the analysis result acquisition unit 22 generates an analysis result including the position, type, and size of the lesion in the lung field as contents indicating the findings. Specifically, as an analysis result, the analysis result acquisition unit 22 acquires the contents of “right lung” and “upper lobe” as the position of the lesion, the contents of “nodule” as the type of the lesion, the contents of “25×21 mm” as the size of the lesion. In a case where a lesion is found, the analysis result acquisition unit 22 may include information, which indicates the storage location of the analyzed medical image in the image database 6, in the analysis result. The information indicating the storage location is used to link an interpretation report as will be described later.

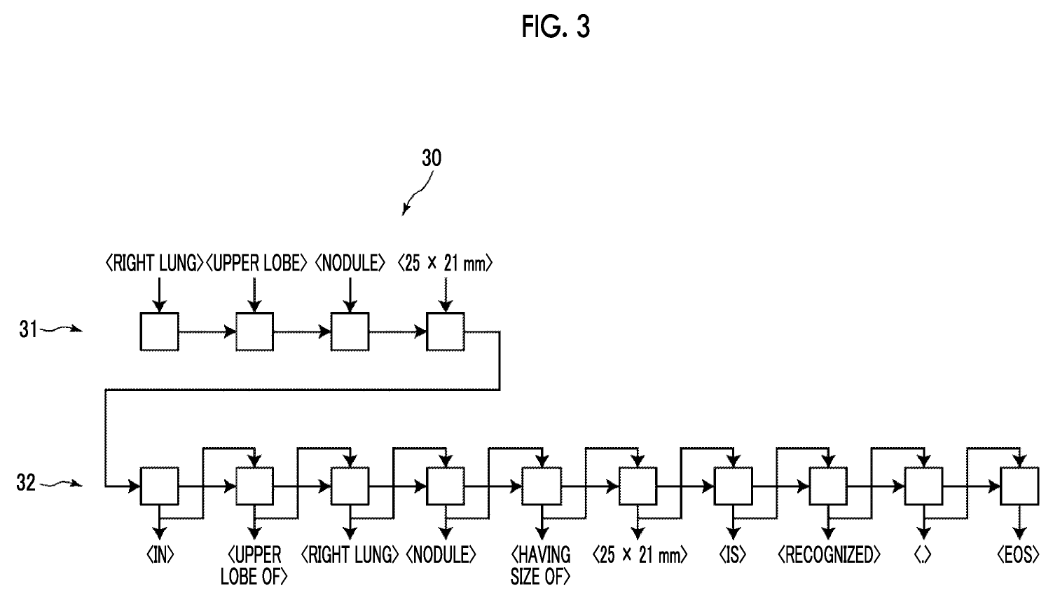

The document creation unit 23 creates an interpretation report based on the keyword input through the input receiving unit 21 and the analysis result acquired by the analysis result acquisition unit 22. In the present embodiment, the document creation unit 23 first creates an interpretation report using the contents indicating the findings included in the analysis result acquired by the analysis result acquisition unit 22. The document creation unit 23 is a recurrent neural network in which learning is performed so as to create a sentence from the input characters. FIG. 3 is a diagram showing the schematic configuration of a recurrent neural network. As shown in FIG. 3, a recurrent neural network 30 includes an encoder 31 and a decoder 32. A keyword or contents included in the analysis result are input to the encoder 31. For example, contents of “right lung”, “upper lobe”, “nodule”, and “25×21 mm” of the analysis result are input to the encoder 31. The decoder 32 is learned so as to make character information into a sentence, and creates a sentence from the character information of the input contents. Specifically, an interpretation report of “in the upper lobe of the right lung, a nodule having a size of 25×21 mm is recognized” is created from the character information of the contents of “right lung”, “upper lobe”, “nodule” and “25×21 mm” described above. In FIG. 3, “EOS” indicates the end of the sentence (End Of Sentence).

The document creation unit 23 also creates an interpretation report based on the keyword received by the input receiving unit 21 as will be described later. The creation of an interpretation report based on the keyword will be described later.

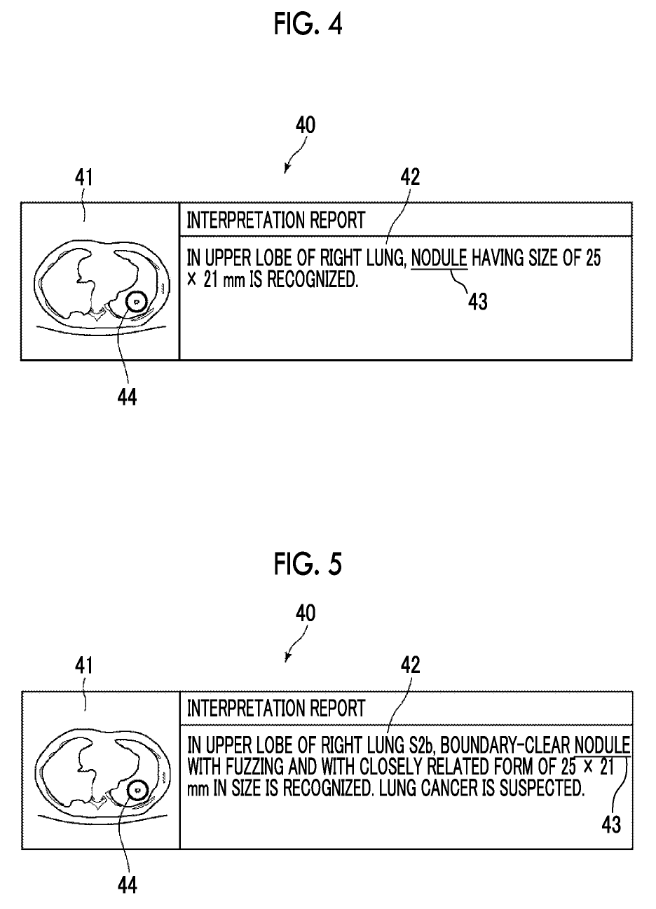

The display control unit 24 displays an interpretation report creation screen including a medical image and an interpretation report on the display unit 14. FIG. 4 is a diagram showing an interpretation report creation screen. As shown in FIG. 4, an interpretation report creation screen 40 has a medical image 41, for which an interpretation report is to be created, and a creation region 42 where inputting for creating an interpretation report is performed. In the creation region 42, an interpretation report of “in the upper lobe of the right lung, a nodule having a size of 25×21 mm is recognized” created by the document creation unit 23 is inserted. Underline is given to the character of “nodule” in this sentence, and a link 43 to the medical image for which an interpretation report is to be created is generated. The operator can display the medical image on the display unit 14 in an enlarged manner by selecting the link 43. A circle 44 is added to the medical image 41 in order to emphasize the portion of the nodule. In a case where an interpretation report using a keyword is newly created as will be described later, the newly created interpretation report is displayed in the creation region 42.

Here, it is assumed that, as a result of interpretation of the medical image 41 by the operator who observes the interpretation report creation screen 40 shown in FIG. 4, findings insufficient in the interpretation report created by the document creation unit 23 are input. For example, it is assumed that “right lung”, “S2b”, “fuzzing”, “boundary clear”, “frosted glass”, and “similar circle” indicating the findings of the medical image and keywords indicating the findings of “lung cancer” are input by voice through the input unit 15.

The input receiving unit 21 converts the voice of the input keyword into a keyword of text, and inputs the keyword to the document creation unit 23. The document creation unit 23 creates a new interpretation report based on the input keyword and the contents included in the analysis result. For example, the input keyword is added to the interpretation report of “in the upper lobe of the right lung, a nodule having a size of 25×21 mm is recognized”, thereby newly creating an interpretation report of “in the upper lobe of the right lung S2b, a boundary-clear nodule with fuzzing and with a closely related form of 25×21 mm in size is recognized. Lung cancer is suspected”. The display control unit 24 displays the newly created interpretation report on the display unit 14. FIG. 5 is a diagram showing an interpretation report creation screen on which a new interpretation report is displayed.

In a case where the operator further inputs a keyword in a state in which a new interpretation report is displayed as described above, the input receiving unit 21 receives an input of the new keyword, the document creation unit 23 further creates a new interpretation report based on the newly input keyword and the contents included in the analysis result, and the display control unit 24 further displays a new interpretation report on the display unit 14.

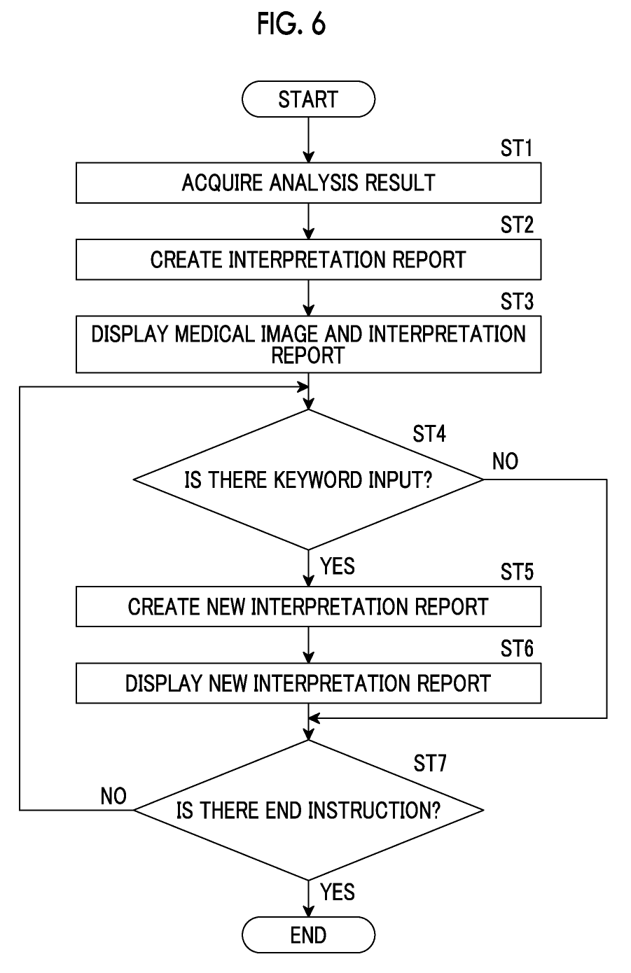

Next, a process performed in the present embodiment will be described. FIG. 6 is a flowchart showing the process performed in the present embodiment. In a case where a medical image to be interpreted is input to the interpretation WS 3 and an instruction to create an interpretation report is given by the operator, the process is started, and the analysis result acquisition unit 22 acquires the analysis result of the medical image (step ST1). Then, the document creation unit 23 creates an interpretation report on the medical image first based on the contents included in the analysis result (step ST2), and the display control unit 24 displays the medical image and the interpretation report on the display unit 14 (step ST3).

Then, the input receiving unit 21 determines whether or not a keyword indicating the findings based on the displayed medical image has been input (step ST4). In a case where there is an input of a keyword and step ST4 is positive, the document creation unit 23 creates a new interpretation report based on the keyword and the analysis result (step ST5). In a case where step ST4 is negative, the process proceeds to step ST7 to be described later. The display control unit 24 displays the newly created interpretation report on the display unit 14 (step ST6).

Then, the input receiving unit 21 determines whether or not there is an end instruction (step ST7). In a case where step ST7 is negative, the process returns to step ST4. In a case where step ST7 is positive, the process is ended.

As described above, according to the present embodiment, an input of a keyword indicating the findings based on the displayed medical image is received, the analysis result of the medical image is acquired, and a medical document relevant to the medical image is created based on the keyword and the analysis result. In this manner, in a case where the operator inputs only a keyword, a medical document including both the analysis result and the operator's recognition is created. Therefore, according to the present embodiment, it is possible to reduce the burden on the operator in a case where a medical document is created. As a result, the operator can efficiently create a medical document. Since the manner of expressing a medical document differs depending on the operator who creates the medical document, there is a variation in a document for each operator. In this manner, in a case where there is a variation in a document, it may be difficult to read a document or it may be difficult to reuse a document. According to the present embodiment, since it is possible to suppress a variation in a created medical document, it is possible to create a medical document that is easy to read and easy to reuse.

In the embodiment described above, the document creation unit 23 creates one interpretation report for the analysis result of one medical image, but the invention is not limited thereto. The document creation unit 23 may create a plurality of candidates for an interpretation report (hereinafter, referred to interpretation report candidates) for one medical image. In this case, the display control unit 24 displays a plurality of interpretation report candidates on the display unit 14.

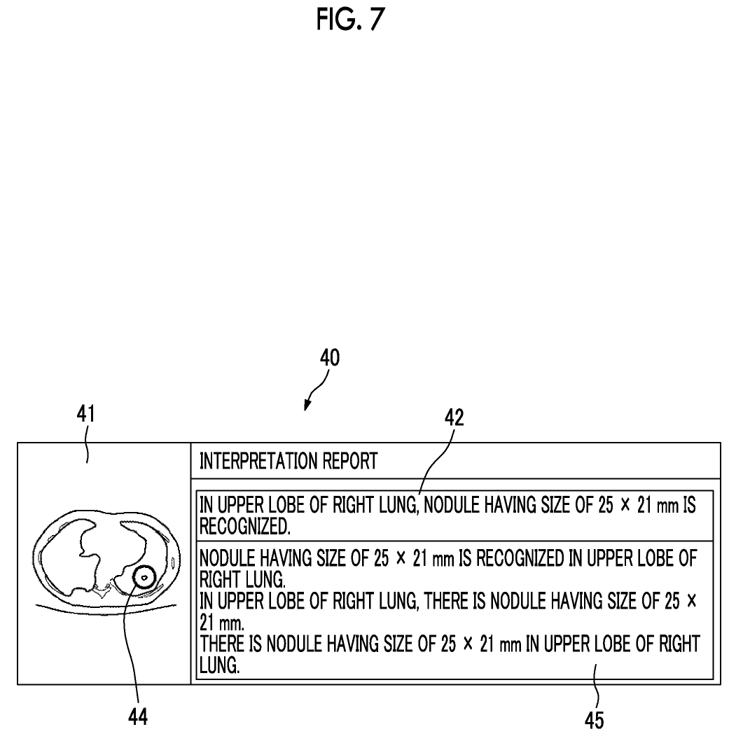

FIG. 7 is a diagram showing a state in which a plurality of interpretation reports are displayed on the display unit 14. As shown in FIG. 7, an interpretation report region 45 for displaying a plurality of interpretation report candidates is displayed in the creation region 42 of the interpretation report. Four types of interpretation report candidates are displayed in the interpretation report region 45. Specifically, interpretation report candidates of “in the upper lobe of the right lung, a nodule having a size of 25×21 mm is recognized”, “a nodule having a size of 25×21 mm is recognized in the upper lobe of the right lung”, “in the upper lobe of the right lung, there is a nodule having a size of 25×21 mm”, and “there is a nodule having a size of 25×21 mm in the upper lobe of the right lung” are displayed.

In this case, in a case where the operator selects a desired interpretation report candidate from a plurality of interpretation report candidates displayed in the interpretation report region 45 using the input unit 15, the display control unit 24 inserts the selected interpretation report candidate into the creation region 42. For example, in a case where the operator selects “in the upper lobe of the right lung, a nodule having a size of 25×21 mm is recognized” among the four types of interpretation report candidates, the selected interpretation report candidate is inserted into the creation region 42 as an interpretation report as shown in FIG. 4. In this manner, by receiving the selection of an interpretation report candidate, which needs to be input, among a plurality of interpretation report candidates, the operator can create an interpretation report having the desired contents.

In the embodiment described above, the analysis result acquisition unit 22 of the medical document creation support apparatus 10 in the interpretation WS 3 analyzes the medical image. However, an external analysis server or the like may analyze a medical image and acquire the analysis result. In this case, the analysis result acquisition unit 22 acquires the analysis result acquired from the outside.

In the embodiment described above, in a case where the interpretation report as a medical document is created, the invention is applied. However, it is needless to say that the invention can be applied even in the case of creating medical documents other than the interpretation report, such as an electronic medical record and a diagnosis report.

In the embodiment described above, a keyword indicating the findings is input by voice. However, the invention is not limited thereto, and it is needless to say that a keyword may be input using a keyboard or the like.

What is claimed is:

1. A medical document creation support apparatus, comprising:
an input receiving unit that receives an input of a keyword indicating findings based on a displayed medical image;
an analysis result acquisition unit that acquires an analysis result of the medical image; and
a document creation unit that creates a medical document relevant to the medical image based on the keyword and the analysis result.

2. The medical document creation support apparatus according to claim 1,
wherein the analysis result acquisition unit acquires the analysis result by analyzing the medical image.

3. The medical document creation support apparatus according to claim 1,
wherein the analysis result includes contents indicating findings based on the medical image, and
the document creation unit creates the medical document based on the keyword and the contents.

4. The medical document creation support apparatus according to claim 1, further comprising:
a display control unit that displays the medical image and the medical document on a display unit.

5. The medical document creation support apparatus according to claim 4,
wherein the input receiving unit receives an input of an additional keyword for the displayed medical document, and
the document creation unit creates the medical document again based on the additional keyword in addition to the keyword and the analysis result.

6. The medical document creation support apparatus according to claim 4,
wherein the document creation unit creates a plurality of medical document candidates for one medical image,
the display control unit displays the plurality of medical document candidates on the display unit, and
the document creation unit further creates the medical document based on a medical document candidate selected from the plurality of medical document candidates.

7. The medical document creation support apparatus according to claim 1,
wherein the medical document is an interpretation report on the medical image.

8. The medical document creation support apparatus according to claim 1,
wherein the medical document includes a link to the medical image.

9. The medical document creation support apparatus according to claim 1,
wherein the input receiving unit receives an input of a keyword of voice indicating the findings and converts the keyword of the voice into text.

10. A medical document creation support method, comprising:
receiving an input of a keyword indicating findings based on a displayed medical image;
acquiring an analysis result of the medical image; and
creating a medical document relevant to the medical image based on the keyword and the analysis result.

11. A non-transitory computer-readable storage medium that stores a medical document creation support program causing a computer to execute:
a step of receiving an input of a keyword indicating findings based on a displayed medical image;
a step of acquiring an analysis result of the medical image; and
a step of creating a medical document relevant to the medical image based on the keyword and the analysis result.
